(19) United States
(12) Patent Application Publication
(10) Pub. No.: US 2026/0000617 A1
(43) Pub. Date: Jan. 1, 2026

(54) ORAL BIOLOGIC MACROMOLECULE DELIVERY SYSTEM

(71) Applicant: NUTRITION & BIOSCIENCES USA 1, LLC, Wilmington, DE (US)

(72) Inventors: True L. Rogers, Midland, MI (US); Kevin P. O'Donnell, Midland, MI (US); Dean Lee, Midland, MI (US); Susan L. Jordan, Wilmington, DE (US); Joshua S. Katz, Wilmington, DE (US); Mark Dreibelbis, Midland, MI (US); Stephanine Robart, Midland, MI (US); Thomas R. Watson, Midland, MI (US); Miriam Carnovale, Wilmington, DE (US); Andrew Horton, Midland, MI (US)

(21) Appl. No.: 19/125,143

(22) PCT Filed: Nov. 3, 2023
(86) PCT No.: PCT/US2023/078593

§ 371 Date: Apr. 28, 2025

Related U.S. Application Data
(60) Provisional application No. 63/382,195, filed on Nov. 3, 2022.
(60) Provisional application No. 63/382,537, filed on Nov. 7, 2022.

(30) Foreign Application Priority Data
Nov. 17, 2022 (EP) 22208010.3

(51) Int. Cl. A61K 9/20; A61K 9/00; A61K 38/26; A61K 38/40; A61K 38/46

(52) U.S. Cl. CPC A61K 9/2054; A61K 9/0053; A61K 38/26; A61K 38/40; A61K 38/465

(57) ABSTRACT

The present invention relates to pharmaceutical dosage forms suitable for gastrointestinal delivery, as well as to methods for the treatment of conditions suitable for treatment by gastrointestinal delivery.

### FIELD OF THE INVENTION

The present invention relates to pharmaceutical dosage forms suitable for gastrointestinal delivery, as well as to methods for the treatment of conditions suitable for treatment by gastrointestinal delivery.

### BACKGROUND OF THE INVENTION

When a biomacromolecule—such as a protein or peptide—is orally administered, the body processes it as a nutrient, typically digesting it down to dipeptides, tripeptides, and amino acids, which are then absorbed as nutrients. If this occurs, the biomacromolecule—if intended for therapeutic effect—will no longer be in its intact native conformation to exert the intended effect. Hence, the biomacromolecule must be sufficiently protected from digestion in the gastrointestinal tract (GIT) and sufficiently retained in its active native conformation in order to retain the ability to have its intended effect, whether that be locally in the GIT for topical effect on the GIT mucosal membrane or for systemic absorption across the GI membrane into the bloodstream for intended effect.

Ideally, the composition exhibits high surface area-to-volume (SA/Vol) ratio for greater contact surface area with the GIT luminal folds, rather than being concentrated within a single, large, low surface area contact point, as is the case with a standard-sized larger tablet.

Greater contact surface area within the GIT luminal folds could reduce adverse effects (GI membrane irritation, ulceration, etc.) and increase contact points with the GIT mucosal membrane for greater efficacy of delivery. Attaining higher SA/Vol ratio of the dosage form would facilitate greater contact surface area.

The disadvantage of greater SA/Vol ratio, however, would be greater exposure of the biomacromolecule active pharmaceutical ingredient (API) to digestive mechanisms (pH, enzymes, microbes) in the GIT. Increasing SA/Vol ratio would typically mean that GI fluid would more rapidly permeate into the composition, consequently digesting a greater portion of the biomacromolecule API dosage within a given timeframe. Increasing SA/Vol ratio without increasing macromolecular digestion would be ideal but is counterintuitive. This present invention breaks that opposing directionality paradigm.

### SUMMARY OF THE INVENTION

It is an object of embodiments of the invention to provide a pharmaceutical solid dosage form for the treatment of a condition by gastrointestinal delivery of one or more therapeutically active biomacromolecules.

The present invention relates in a broad aspect to a multi-site microenvironment concept in the form of a solid dosage form for effective delivery of biomacromolecules through the gastrointestinal system.

Accordingly, in a first aspect the present invention relates to a pharmaceutical solid dosage form for the treatment of a condition by gastrointestinal delivery comprising multiple single units, each single unit comprising:
a) one or more therapeutically active biomacromolecules;
b) one or more water-soluble polymers in an amount of not more than about 50 weight %;
c) a small-molecule weak acid (WA), weak acid surfactant (WAS), or salt thereof in an amount of not more than about 75 weight %;
each single unit having a surface area to volume (SA/Vol) ratio greater than 1.0 mm-1
and a single-unit density greater than 1.0 g/cm³; the solid dosage form comprising the therapeutically active biomacromolecule in a therapeutically effective amount derived from the combined multiple amount of each single unit.

In a second aspect the present invention relates to a method for treating a subject in need of a biomacromolecule as defined herein, the method comprising (a) providing a solid oral dosage form according to the present invention, and (b) administering orally to a patient this solid oral dosage form. In some embodiments the solid dosage form provides a pharmacokinetic profile of the active biomacromolecule with a T lag greater than 1.0 h and less than 16 h post-administration and a T max greater than (T lag+0.5 h) and less than 20 h post-administration.

In a third aspect the present invention relates to a process for the preparation of a pharmaceutical solid dosage form as defined herein, which process comprises the steps of providing the components a), b) and c), and formulating the dosage form into a tablet or a capsule, such as by tableting, direct compression tableting, dry granulation followed by tableting, roller compaction followed by tableting, dry powder layering, pelletization, slugging; the process optionally comprising a step of encapsulation.

### DETAILED DESCRIPTION OF THE INVENTION

The present invention is directed to a composition which has high SA/Vol ratio for greater contact surface area with the GIT luminal folds while also protecting the biomacromolecule API from digestion to an extent similar to that of a lower SA/Vol, standard-sized tablet. I.e. the inventive composition has greater SA/Vol ratio than a standard-sized tablet for greater contact surface area, without sacrificing macromolecular API protection from digestive mechanisms, which would otherwise occur with high SA/Vol. Thus, the intact biomacromolecule API will have more time and contact surface area for either localized action or systemic absorption, whichever is called for. The opposing directionality paradigm is thus broken where high SA/Vol is attained without the disadvantage of increasing susceptibility of the biomacromolecule API to digestive mechanisms.

The composition should also exhibit single-unit density sufficient to facilitate sinking of the composition down into the GIT luminal folds.

It is to be understood that the bulkier biomacromolecule diffuses more slowly with a Pₐₚₚ coefficient of ^{˜}10⁻⁹ relative to the Pₐₚₚ coefficient of a small molecule, which is ^{˜}10⁻⁴. i.e. a small molecule has ^{˜}5 fold faster Pₐₚₚ coefficient. Formulating a water-soluble polymer into the single units comprising the composition can form a hydrogel, causing the small-molecule weak acid (WA), weak acid surfactant (WAS), or salt thereof to diffuse more slowly, more comparable to that of the biomacromolecule, retaining the small-molecule weak acid (WA), weak acid surfactant (WAS), or salt thereof in close proximity to the biomacromolecule and creating a microenvironment around each unit of the composition to sufficiently protect the biomacromolecule from GIT digestive mechanisms for up to at least 1 h.

What results are multiple units of the composition within a particular location of the GIT, each with its own microenvironment, that functions to sufficiently protect the biomacromolecule from digestion for up to at least 1 h for greater availability of the intact biomacromolecule API within the GIT lumen for its intended effect.

The volume of fluid in the GIT at any given time is relatively small, but there is significant turnover of fluids flushing through the GIT and being reabsorbed. The solid dosage form according to the present invention facilitates retention of a greater portion of the faster-diffusing small-molecule weak acid (WA), weak acid surfactant (WAS), or salt thereof in close proximity to the slower-diffusing biomacromolecule for greater protection from digestion, even in the presence of high GIT fluid turnover.

### Biomacromolecules

As used herein a “therapeutically active biomacromolecule” refers to a molecule with a molecular weight equal to or higher than about 500 Da, which has a therapeutic effect in a subject in need thereof. It is thus to be understood that the present invention is not intended for small organic molecule API of sole use, however one or more small organic molecule APIs could be combined with one or more biomacromolecules. Suitable therapeutically active biomacromolecules within the present invention include but is not limited to oligonucleotides, DNA fragments, RNA fragments, messenger RNA, small interfering RNA, modified RNA, oligopeptides, peptides and polypeptides from smaller peptides to larger antibodies and multi-subunit proteins including but not limited to synthetic polypeptides, hormones, insulins, growth factors, monoclonal antibodies, fusion proteins, enzymes, therapeutic enzymes, bispecific antibodies, multi-specific antibodies, antibody fragments, interleukins, cytokines, antibody-drug conjugates, glycoproteins, and viral proteins such as peptides selected from the group consisting of leuprolide, insulin, vasopressin, calcitonin, calcitonin gene-related peptide, desmopressin, gonadotrophin releasing hormone (GnRH), luteinizing hormone-releasing factor, adrenocorticotropin, enkephalin, glucagon, glucagon-like peptide-1, glucagon-like peptide-2, somatostatin, gastrin, glucose insulinotropic polypeptide, peptide yy, amylin, islet amyloid polypeptide, linaclotide, octreotide, semaglutide, liraglutide, tirzepatide, dulaglutide, exenatide, lixisenatide, ecnoglutide, oxytocin, and 2,6-dimethyltyrosine-D-arginine-phenylalanine-lysine amide, or a polypeptide or protein selected from the group consisting of an antibody, vaccine, lactoferrin, parathyroid hormone, growth hormone, human growth hormone, cytokine, interferon, interleukin or antagonists thereof, such as any of IL1-40, such as IL1, IL2, IL10, IL12, IL19, IL21, IL23, IL26, IL27, IL28, IL29, IL36, IL37, IL38, IL39, IL40, lysozyme, β-casein, albumin, «-1 antitrypsin, antithrombin III, collagen, factor VII, factor VIII, factor IX, factor X, fibrinogen, insulin, protein C, erythropoietin (EPO), granulocyte colony-stimulating factor (G-CSF), granulocyte macrophage colony-stimulating factor (GM-CSF), tissue-type plasminogen activator (tPA), somatotropin, integrins, alpha-4, beta-7 integrins, chymotrypsin, lipase, pancrelipase, amylase, and protease, monoclonal antibodies, such as adalimumab, tofacitinib, foralumab, bevacizumab, rituximab, trastuzumab, denosumab, ranibizumab, tocilizumab, certolizumab, golimumab, secukinumab, Griffithsin, alpha 1,2-fucosidase, xylanase, phytase, and tumor necrosis factor (TNF). As used herein a peptide refers to a collection of amino acids linked together through peptide (amide) bonds. A polypeptide refers to longer chains of amino acids usually with more than 50 amino acids, whereas oligopeptides refers to chains of fewer than 20 amino acids. The terms peptide, oligopeptide and polypeptide are intended to include both branched and continuous unbranched chains of amino acids. Proteins as used herein refers to chains of amino acids, such as peptides or polypeptides in any primary, secondary, tertiary and quaternary structure and potentially encompassing any posttranslational modification such as a phosphorylation, glycosylation, ubiquitination, nitrosylation, methylation, acetylation, lipidation and proteolysis.

In some embodiments, the therapeutically active biomacromolecule comprises an enzyme. In some embodiments, the enzyme comprises a lipase, protease, amylase, enterokinase or carbohydrate enzyme. In some such embodiments, the enzyme comprises a lipase. In some embodiments, the enzyme comprises a lipase. In some embodiments, the enzyme comprises a co-lipase. In some embodiments, the enzyme comprises phospholipase A1 or phospholipase A2. In some embodiments, the enzyme comprises esterase. In some embodiments, the enzyme comprises pancreatic lipase-related protein 2. In some embodiments, the enzyme comprises gastric lipase. In some embodiments, the enzyme comprises a protease. In some embodiments, the protease comprises trypsin, chymotripsin, carboxypolypeptidase, an elastase, a nuclease, pepsin or a subtilisin. In some embodiments, the enzyme comprises an amylase. In some such embodiments, the amylase comprises an alpha-amylase, beta-amylase or gluco-amylase. In some embodiments, the enzyme comprises enterokinase. In some embodiments, the enzyme comprises a carbohydrate enzyme. In some such embodiments, the enzyme comprises a cellohydrolases, beta-glucosidase, lactase, galactase, trehelase, mannanase, alpha-glucosidase, sucrase, isomaltase or xylanase.

### Water-Soluble Polymers

As used herein “water-soluble polymers” refers to any water-soluble polymer known to the person skilled in the art, such as ones suitable for pharmaceutical use. Suitable known water-soluble polymers that may be used according to the present invention includes cellulose derivative polymers, such as the ones selected from the list consisting of hydroxypropyl methylcellulose (HPMC), methylcellulose (MC), carboxymethylcellulose (CMC), such as sodium carboxymethylcellulose, hydroxypropyl cellulose (HPC), hydroxyethyl cellulose (HEC), hydroxyethyl methylcellulose (HEMC). Suitable known water-soluble polymers also include alginate, such as sodium alginate, carrageenan, pectin, chitosan, trimethyl chitosan, hyaluronic acid, polycarbophil, carbomer, polyethylene oxide, polyvinylpyrrolidone and copolymers thereof, and methacrylic acid derivative polymers, and derivatives and salts thereof. The water-soluble polymer can serve such a role as hydrogel-former.

### Cellulose or Cellulose Derivative Polymers

Any suitable cellulose or cellulose derivative polymer may be used according to the present invention. The person skilled in the art will know these suitable polymers.

Suitable cellulose or cellulose derivative polymer used according to the present invention includes cellulose, microcrystalline cellulose (MCC), low-viscosity hydroxypropylcellulose (HPC), ethylcellulose (EC), methylcellulose (MC), carboxymethyl cellulose (CMC), and hydroxypropyl methylcellulose (HPMC), such as hypromellose 2910 (7-12% HP, 28-30% methoxy), hypromellose 2906 (4-7.5% HP, 27-30% methoxy), Hypromellose 2208 (4-12% HP, 19-24% methoxy), Hypromellose 1828 (23-32% HP, 16.5-20% methoxy). Commercially available microcrystalline cellulose (MCC) includes Avicel® from IFF and Emcocel from JRS Pharma. Commercially available carboxymethyl cellulose (CMC) includes TEXTURECEL™ from IFF, Celetec™ from CPKelco, Aqualon™ from Ashland, Rheoflo® from USK Kimya A.S. and Akucell® from Nouryon (formerly AkzoNobel). Commercially available methylcelluloses and hydroxypropyl methylcelluloses include Japanese Pharmacopoeia METOLOSE and PHARMACOAT (trademark) series and METOLOSE and PHARMACOAT series for food additives from Shin-Etsu Chemical Co., Ltd., AnyCoat-C or AnyAddy (trademark) series from Lotte (formerly Samsung) Fine Chemicals Co., Ltd., METHOCEL (trademark) series from International Flavors & Fragrances (IFF) (formerly DOW Chemical Company), and Benecel (trademark) series from Ashland.

Methylcellulose is one suitable cellulose derivative polymer to the present invention. Methylcellulose has anhydroglucose units joined by 1-4 linkages. Each anhydroglucose unit contains hydroxyl groups at the 2, 3, and 6 positions. Partial or complete substitution of these hydroxyls with methoxyl groups creates methylcellulose. For example, treatment of cellulosic fibers with caustic solution, followed by a methylating agent, yields cellulose ether substituted with one or more methoxyl groups. If not further substituted with other alkyls, this cellulose ether is known as methylcellulose. Methylcellulose is characterized by the weight percent of methoxyl groups. By convention, the weight percent is an average weight percentage based on the total weight of the cellulose repeat unit, including all substituents. The content of the methoxyl group is reported based on the mass of the methoxyl group (i.e., —OCH₃). The determination of the % methoxyl in methylcellulose (MC) polymer is carried out according to the United States Pharmacopeia (USP 37, “Methylcellulose”, pages 3776-3778). The % methoxyl can be converted into degree of substitution (DS) for methyl substituents, DS (methyl). DS (methyl), also designated as DS (methoxyl), of a methylcellulose is the average number of OH groups substituted with methyl groups per anhydroglucose unit.

Hydroxyalkyl methylcellulose is another suitable cellulose derivative polymer to the present invention. Hydroxyalkyl methylcellulose is a cellulose ether having anhydroglucose units joined by 1-4 linkages and having both methyl groups and hydroxyalkyl groups. The hydroxyalkyl groups can be the same or different from each other. Preferably the cellulose ether comprises one or two kinds of hydroxyalkyl groups, more preferably one or more kinds of hydroxy-C1-3-alkyl groups, such as hydroxypropyl and/or hydroxyethyl. Useful optional alkyl groups are, e.g., ethyl or propyl. Preferred ternary cellulose ethers are ethyl hydroxypropyl methyl celluloses, ethyl hydroxyethyl methyl celluloses, or hydroxyethyl hydroxypropyl methyl celluloses. Preferred cellulose ethers are hydroxyalkyl methyl celluloses, particularly hydroxy-C1-3-alkyl methyl celluloses, such as hydroxypropyl methylcelluloses or hydroxyethyl methylcelluloses.

The cellulose ether has a DS (methyl) of from 1.2 to 2.2, preferably 1.2 to 1.6. The degree of the methyl substitution, DS (methyl), of a cellulose ether is the average number of OH groups substituted with methyl groups per anhydroglucose unit. For determining the DS (methyl), the term “OH groups substituted with methyl groups” does not only include the methylated OH groups directly bound to the carbon atoms of the cellulose backbone but also methylated OH groups that have been formed after hydroxyalkylation.

The cellulose ether has an MS (hydroxyalkyl) of 0.05 to 1.00, preferably 0.08 to 0.80, more preferably 0.12 to 0.70, even more preferably 0.15 to 0.60, most preferably 0.20 to 0.40, and particularly 0.25 to 0.35. The degree of the hydroxyalkyl substitution is described by the MS (molar substitution). The MS (hydroxyalkyl) is the average number of hydroxyalkyl groups which are bound by an ether bond per mole of anhydroglucose unit. During the hydroxyalkylation multiple substitutions can result in side chains.

For hydroxypropyl methylcellulose, the determination of the % methoxyl and % hydroxypropoxyl in hydroxypropyl methylcellulose is carried out according to the United States Pharmacopeia (USP 43). The values obtained are % methoxyl and % hydroxypropoxyl. These are subsequently converted into degree of substitution (DS) for methyl substituents and molar substitution (MS) for hydroxypropyl substituents. Residual amounts of salt have been taken into account in the conversion. The DS (methyl) and MS (hydroxyethyl) in hydroxyethyl methylcellulose is effected by Zeisel cleavage with hydrogen iodide followed by gas chromatography. (G. Bartelmus and R. Ketterer, Z. Anal. Chem. 286 (1977) 161-190).

The viscosity of the cellulose ether of the present invention is determined as a 2% by weight solution in water using Brookfield rotational viscometer or a Ubbelohde tube according to the United States Pharmacopeia (USP 43). The solution for the viscosity measurements of the cellulose ether is prepared by adding the appropriate amount of cellulose ether powder to the appropriate amount of water in order to achieve a concentration of 2% while stirring with an overhead lab stirrer at elevated temperature. Afterwards the solution is equilibrated to lower temperature as specified in USP 43.

The solution for the viscosity measurements of the sodium carboxymethylcellulose (CMC e.g. TEXTURACEL™ 20000 PA 07) are prepared by adding the appropriate amount of the CMC powder to the appropriate amount of water in order to achieve a concentration of 1 or 2% according to the USP 43 while stirring with an overhead lab stirrer at ambient temperature for at least 1 h. The viscosity is measured according to USP 43.

### Alginate and Salts Thereof.

Alginates, derived from, inter alia, brown seaweeds are linear, unbranched bio-polymers consisting of (1-4)-linked β-D-mannuronic acid (M) and α-L-guluronic acid (G) residues. Alginates are not random copolymers but consist of blocks of similar and alternating sequences of residues, for example, MMMM, GGGG, and GMGM. In extracted form alginate absorbs water quickly. The physical properties of alginates may depend on the relative proportion of the M and G blocks. Gel formation at neutral pH requires a calcium source to provide calcium ion to interact with G-blocks. The greater the proportion of these G-blocks, the greater the gel strength.

“Alginate” is the term usually used for the salts of alginic acid, but it can also refer to all the derivatives of alginic acid and alginic acid itself; Alginate is present in the cell walls of brown algae as the calcium, magnesium and sodium salts of alginic acid. Dry, powdered, sodium alginate or potassium alginate may be obtained from an extraction process of this brown algae. The seaweed residue is then removed by filtration and the remaining alginate may then be recovered from the aqueous solution.

Another way to recover the alginate from the initial extraction solution is to add a calcium salt. This causes calcium alginate to form with a fibrous texture; it does not dissolve in water and can be separated from it. The separated calcium alginate is suspended in water and acid is added to convert it into alginic acid.

Alginates suitable for use in the practice of this invention will typically have a molecular weight such that they exhibit a viscosity in the range of 5-1,000 mPa·s. when measured at 2 wt % at 20° C. using rheometer setup with cup and bob geometry at a shear rate of 10 s⁻¹. In some embodiment, such alginates will exhibit a viscosity of between 6 and 600 mPa·s, such as between 7 and 500 mPa·s, or between 8 and 500 mPa·s when so measured. In some other embodiment, such alginates will exhibit a viscosity of between 8 and 400 mPa·s, such as between 8 and 300 mPa·s, such as between 9 and 200 mPa·s, or between 10 and 100 mPa·s when so measured. In some embodiments according to the present invention, a high G type alginate is used. A high G type alginate means that the alginate(s) employed in the practice of the present invention possess an average of at least 50 percent adjacent G units. In some embodiments the alginate will possess an average of at least 52 percent adjacent G units; in other embodiments such alginate will possess an average of at least 55 percent or more of adjacent G units, and in other embodiments such alginate will possess an average of at least 60, 65, or 70 percent or more of adjacent G units, as such higher the content of adjacent G units may result in improved product textures.

According to the present invention, alginate, such as alginic acid or an alginate salt is present in the amount of not more than 30% (w/w) based on the total weight of the final composition. In the present invention, alginate refers to any alginic acid or an alginate salt, such as sodium alginate, magnesium alginate, potassium alginate, triethanolamine alginate or propylene glycol monoglycolate.

Another suitable water-soluble polymer that may be used according to this specification is carrageenan. As will be appreciated by one skilled in the art, ingredients obtained from seaweed of the class of Rhodophyta will contain carrageenan. Carrageenan refers to a family of linear sulfated polysaccharides that are extracted from red edible seaweeds. Carrageenan is a high-molecular-weight polysaccharide made up of repeating galactose units and 3,6 anhydrogalactose (3,6-AG), both sulfated and nonsulfated. The units are joined by alternating α-1,3 and β-1,4 glycosidic linkages.

Suitable carrageenan products for pharmaceutically acceptable blends or pharmaceutical formulations according to this specification include various commercial carrageenans, such as Gelcarin® carrageenan and Viscarin® carrageenan grades, such as Gelcarin® GP-379NF, Viscarin® 101, Viscarin® GP-328NF, Viscarin® GP-209NF, Viscarin® GP109 Gelcarin® GP911, Gelcarin® GP-812NF (IFF Nutrition & Biosciences).

Another suitable water-soluble polymer that may be used according to the present invention is pectin. The term “pectin” is to be understood as a water-soluble form of pectic substance obtained by extraction of pectin from a plant material. Pectin has a structure comprising blocks of linear galacturonan chains (polymer of α-(1-4)-linked-D-galacturonic acid) which are interrupted with rhamno-galacturonan backbones (polymers of the repeating disaccharide α-(1-4)-D-galacturonic acid-α-(1-2)-L-rhamnose), which often have side chains of polymeric arabinogalactans glycosidically linked to the 0-3 or O-4 positions of L-rhamnose. The galacturonan sequences can have D-xylose and D-apiose glycosidically linked to their O-2 or O-3 positions, which also can be substituted with ester-linked acetyl groups. The long chains of α-(1-4)-linked D-galacturonic acid residues are commonly referred to as “smooth regions”, whereas the highly branched rhamnogalacturonan regions are commonly referred to as the “hairy regions”.

Pectin is a commonly and important polysaccharide with applications in both foods and pharmaceuticals and many commercial sources exist. Most sources of commercial pectin products are citrus peel and apple pomace in which protopectin represents 10-40% by weight of the dry matter.

Pectin is present in almost all higher plants. Some by-products of the food industry are used for pectin extraction, such as citrus peels (by-products of citrus juice production), apple pomace (by-products of apple juice production), beets (by-products of beet sugar industry), slightly extended to Potato fiber, sunflower heads (by-product of oil production) and onions (May 1990, Carbohydr. Polymers, 12:79-99). A typical method for extracting hypermethylated (HM) pectin from pomace or peel is to extract in hot dilute mineral acid at pH 1-3, 50-90° C. for 3-12 hours (Rolin, 2002, in Pectins and their Manipulation; Seymour GB), Knox JP, Blackwell Publishing Ltd, 222-239). The dried citrus peel contains 20-30% pectin (based on dry matter) and the pectin in the dried apple pomace is present in low amounts (10-15%) (Christensen, 1986, Pectins. Food Hydrocolloids, 3, 205-230). Pectin is precipitated by the addition of an alcohol (usually isopropanol but also methanol or ethanol). Finally, the gelatinous material is pressurized, washed, dried and ground (Carbohydr. Polymers, 12:79-99, May 1990). Depending on the process conditions, pectins may be obtained as described in Rolin, 2002, in Pectins and their Manipulation; Seymour G. B., Knox J. P., Blackwell Publishing Ltd, 222-239.

Hypomethylated (LM) pectin can be obtained by de-esterification of hypermethylated (HM) pectin, primarily by controlling the acidity, temperature and time during the extraction process. In order to produce other types of pectin, the ester may be hydrolyzed as a concentrated liquid or in an alcoholic slurry by acid or base before or during extraction, and then separated and dried. When a base is used, the reaction must be carried out in a low temperature and in an aqueous solution to avoid β-eliminating degradation of the polymer (Kravtchenko et al., 1992, Carbohydrate Polymers, 19, 115-124). LM pectin (e.g., potato pectin) can also be extracted with an aqueous chelating agent such as hexametaphosphate (Voragen et al, 1995, in Food polysaccharides and their applications; Stephen A. M., New York: Marcel Dekker Inc, 287-339). The use of pectin methyl esterase (PME) to produce LM pectin can be used as an alternative to chemical extraction (Christensen, 1986, Pectins. Food Hydrocolloids, 3, 205-230). Although commercial LM pectin is almost entirely derived from HM pectin, there is a natural source of LM pectin, such as the mature sunflower head (Thakur et al., 1997, Critical Reviews in Food Science and Nutrition, 37 (I): 47-73). One method of producing pectin is described in International Patent Application WO 2013/109721, wherein citrus peel is treated to obtain homogenized citrus peel, the homogenized citrus peel is washed with an organic solvent, followed by a desolventizing and drying step to recover the fiber-containing pectin product or pectin. In some embodiments, a comminuting or pulverizing step is carried out after the drying step.

Alternatively, a suitable pectin product is obtained according to the process described in U.S. Pat. No. 7,833,558, which patent describes a method of providing a fiber-containing pectin product from a plant material which comprises the steps of (i) providing an in situ reaction system by swelling the plant material in an aqueous solution comprising at least one salt, (ii) subjecting pectin present in the swollen plant material from step (i) to a de-esterification treatment, and (iii) separating the de-esterified fiber-containing pectin product. In some embodiments, the plant material is native pectin-containing plant materials including peels or pulp from citrus fruits, such as lemon, orange, mandarin, lime and grapefruit.

Exemplary commercially available pectin's include, but are not limited to, apple pectin (SIGMA-ALDRICH, product number 93854), citrus peel pectin (SIGMA-ALDRICH, product number P9135), citrus pectin with a degree of esterification of 60% (SIGMA-ALDRICH, product number P9436) and citrus pectin with a degree of esterification of 90% (SIGMA-ALDRICH, product number P9561), GRINDSTED Pectin RS 400, Andre Pectin AP 101, GENUPECTIN B Rapid Set, UNIPECTIN RS 150, Classic AF 101, GRINDSTED Pectin AMD 780, Andre Pectin AP 140, GENUPECTIN JMJ, UNIPECTIN AYD 20, Classic CM 201/203, GRINDSTED Pectin LC 810, Andre Pectin AP 310, GENUPECTIN LM 18 CG, and UNIPECTIN OF 400, Classic AF 701.

### Small-Molecule Weak Acid (WA), Weak Acid Surfactant (WAS)

The solid dosage form according to the present invention comprises a small-molecule weak acid (WA) or weak acid surfactant (WAS). The term “weak acid” is used here in its normal meaning known to the skilled person referring to an acid that partially dissociates into its ions in an aqueous solution or water. Additionally, a small-molecule weak acid (WA) is an acid with a molecular weight less than 500 Da. In some embodiments, the small-molecule weak acid (WA), weak acid surfactant (WAS), or salt thereof has a molecular weight of less than 400 Da, such as less than 300 Da, such as less 200 Da. The difference between a weak acid (WA) and a weak acid surfactant (WAS) is that the WAS will have amphiphilic nature. As chain length increases, a WA can become a WAS. For example, citric acid is considered a branched WA with 6 carbons total and three carboxyl groups There is one carboxyl group at each end of the five-carbon chain and one carboxyl group at the third carbon of the five-carbon chain. Sodium caprylate can be considered either a WA or a WAS and is a linear 8 carbon chain with a carboxyl group at one end. The amphiphilic nature comes from the nonpolar nature of the eight-carbon chain and the polar nature of the carboxyl group. The WA or WAS can serve such roles as buffering agent, surfactant, and/or absorption enhancer.

Suitable small-molecule weak acid (WA), weak acid surfactant (WAS), or salt thereof to be used according to the present invention may be, but are not limited to, any one selected from a carbonic acid, monovalent metal phosphate salts, citric acid, succinic acid, oleic acid, caprylic acid, capric acid, decanoic acid, lauric acid, phosphotidylcholine, salicylic acid, methylsalicylic acid, ethylene diamine tetraacetic acid, acetic acid, cholic acid, deoxycholic acid, glycolic acid, glycocholic acid, glycodeoxycholic acid, taurocholic acid, taurodihydrofusidic acid, sodium caprate, sodium decanoate, sodium caprylate, sodium octanoate, sodium laurate, sodium dodecyl sulfate, dioctyl sodium sulfosuccinate, glyceryl behenate, glyceryl dibehenate, glyceryl monostearate, sodium N-[8-(2-hydroxybenzoyl)aminocaprylate], salcaprozate sodium, SNAC, N-(5-chlorosalicyloyl)-8-aminocaprylic acid, 5-CNAC, N-[10-(2-hydroxybenzoyl)aminocaprate], N-[10-(2-hydroxybenzoyl)aminodecanoate], sodium lauryl sulfate, sodium stearyl fumarate, or sodium deoxycholate.

### Surface Area to Volume (SA/Vol) Ratio and Single-Unit Density

The solid dosage form according to the present invention consists of multiple single units with a relatively high surface area to volume (SA/Vol) ratio for greater contact surface area with the luminal folds of the gastrointestinal tract. The surface area to volume (SA/Vol) ratio as well as the single-unit density may be measured as described in example 1. The terms “minitablet” and “single-unit” are terms used interchangeably.

### Multiple-Unit Bulk Density

The multiple-unit bulk density refers to the density of the combined amount of individual single units. The multiple-unit bulk density is in some embodiments greater than 0.70 g/cm³.

As detailed above the present invention relates to a pharmaceutical solid dosage form for the treatment of a condition by gastrointestinal delivery comprising multiple single units, each single unit comprising:
a) one or more therapeutically active biomacromolecules;
b) one or more water-soluble polymer in an amount of not more than about 50 weight %;
c) a small-molecule weak acid (WA), weak acid surfactant (WAS), or salt thereof in an amount of not more than about 75 weight %;
each single unit having a surface area to volume (SA/Vol) ratio greater than 1.0 mm-1
and a single-unit density greater than 1.0 g/cm³; the solid dosage form comprising the therapeutically active biomacromolecule in a therapeutically effective amount derived from the combined multiple amount of each single unit

In some embodiments this solid dosage form comprises at least 2 single units as defined above, such as at least about 5, 10, 15, 20, 25, 30, 35, 40, 45, 50, 55, 60, 65, 70, 75, 80, 85, 90, 95, 100, 150, 200, or 250 single units.

In some embodiments this solid dosage form comprises not more than about 500 single units, such as not more than about 400 single units, not more than about 300 single units, not more than about 200 single units, not more than about 180 single units, not more than about 160 single units, not more than about 140 single units, not more than about 120 single units, not more than about 100 single units.

In some embodiments this solid dosage form has a multiple-unit bulk density greater than 0.70 g/cm³, such as greater than 0.72 g/cm³, greater than 0.74 g/cm³, greater than 0.76 g/cm³, greater than 0.78 g/cm³, greater than 0.80 g/cm³, greater than 0.82 g/cm³, greater than 0.84 g/cm³, greater than 0.86 g/cm³, greater than 0.88 g/cm³, greater than 0.90 g/cm³, greater than 0.92 g/cm³, greater than 0.94 g/cm³, greater than 0.96 g/cm³, greater than 0.98 g/cm³, greater than 1.00 g/cm³.

In some embodiments the viscosity of the one or more water-soluble polymers is less than 3000 mPa-s (or cP), such as less than about 2800 mPa-s (or cP), such as less than about 2600 mPa-s (or cP), such as less than about 2400 mPa-s (or cP), such as less than about 2200 mPa-s (or cP), such as less than about 2000 mPa-s (or cP), such as less than about 1800 mPa-s (or cP), such as less than about 1600 mPa-s (or cP), such as less than about 1400 mPa-s (or cP).

In some embodiments each single unit has a surface area to volume (SA/Vol) ratio greater than 1.0 mm⁻¹, such as greater than 1.5 mm⁻¹, such as greater than 2.0 mm⁻¹, such as greater than 2.5 mm⁻¹, such as greater than 3.0 mm⁻¹.

In some embodiments each single unit has a single-unit density greater than 1.0 g/cm³, such as greater than 1.1 g/cm³, such as greater than 1.2 g/cm³, such as greater than 1.3 g/cm³.

In some embodiments the one or more water-soluble polymers is present in an amount of not more than about 50%, such as not more than about 45%, such as not more than about 40%, such as not more than about 35%, such as not more than about 30%, such as not more than about 28 weight %, such as not more than about 26 weight %, such as not more than about 24 weight %, such as not more than about 22 weight %, such as not more than about 20 weight %, such as not more than about 18 weight %, such as not more than about 16 weight %, such as not more than about 14 weight %, such as not more than about 12 weight %, such as not more than about 10 weight %.

In some embodiments the small-molecule weak acid (WA), weak acid surfactant (WAS), or salt thereof is present in an amount of not more than about 70 weight %, such as not more than about 65 weight %, such as not more than about 60 weight %, such as not more than about 55 weight %, such as not more than about 50 weight %, such as not more than about 45 weight %, such as not more than about 40 weight %.

In some embodiments the one or more biomacromolecules are independently selected from a protein, a peptide, a polypeptide, an oligopeptide, a synthetic polypeptide, a hormone, an insulin, a growth factor, a monoclonal antibody, a fusion protein, an enzyme, a therapeutic enzyme, a bispecific antibody, a multi-specific antibody, an antibody fragment, an interleukins, a cytokine, an antibody-drug conjugate, a glycoprotein, a viral protein, an oligonucleotide, a DNA fragment, an RNA fragment, messenger RNA, small interfering RNA, modified RNA, or any combination thereof.

In some embodiments the one or more biomacromolecules is combined with one or more small molecule APIs.

In some embodiments the one or more biomacromolecules is a peptide selected from the group consisting of leuprolide, insulin, vasopressin, calcitonin, calcitonin gene-related peptide, desmopressin, gonadotrophin releasing hormone (GnRH), luteinizing hormone-releasing factor, adrenocorticotropin, enkephalin, glucagon, glucagon-like peptide-1, glucagon-like peptide-2, somatostatin, gastrin, glucose insulinotropic polypeptide, peptide yy, amylin, islet amyloid polypeptide, linaclotide, octreotide, semaglutide, liraglutide, tirzepatide, dulaglutide, exenatide, lixisenatide, ecnoglutide, oxytocin, and 2,6-dimethyltyrosine-D-arginine-phenylalanine-lysine amide.

In some embodiments the one or more biomacromolecules is a protein selected from the group consisting of an antibody, vaccine, lactoferrin, parathyroid hormone, growth hormone, human growth hormone, cytokine, interferon, interleukin or antagonist thereof, such as any of IL1-40, such as IL1, IL2, IL10, IL12, IL19, IL21, IL23, IL26, IL27, IL28, IL29, IL36, IL37, IL38, IL39, IL40, lysozyme, β-casein, albumin, α-1 antitrypsin, antithrombin III, collagen, factor VII, factor VIII, factor IX, factor X, fibrinogen, insulin, protein C, erythropoietin (EPO), granulocyte colony-stimulating factor (G-CSF), granulocyte macrophage colony-stimulating factor (GM-CSF), tissue-type plasminogen activator (tPA), somatotropin, integrins, alpha-4, beta-7 integrins, chymotrypsin, lipase, pancrelipase, amylase, and protease, adalimumab, tofacitinib, foralumab, bevacizumab, rituximab, trastuzumab, denosumab, ranibizumab, tocilizumab, certolizumab, golimumab, secukinumab, Griffithsin, alpha 1,2-fucosidase, xylanase, phytase, and tumor necrosis factor (TNF).

In some embodiments the one or more biomacromolecules has a molecular weight higher than about 500 Da, such as higher than about 2000 Da, such as higher than about 3000 Da, such as higher than about 4000 Da, such as higher than about 5000 Da, such as higher than about 6000 Da, such as higher than about 8000 Da, such as higher than about 10 kDa, such as higher than about 20 kDa, such as higher than about 30 kDa, such as higher than about 40 kDa, such as higher than about 50 kDa, such as higher than about 60 kDa, such as higher than about 70 kDa, such as higher than about 80 kDa, such as higher than about 90 kDa, such as higher than about 100 kDa, such as higher than about 110 kDa, such as higher than about 120 kDa, such as higher than about 130 kDa, such as higher than about 140 kDa, such as higher than about 150 kDa.

In some embodiments the solid dosage form further comprises one or more additional ingredients that provide processability, densification, or identification, such as, but not limited to microcrystalline cellulose, mannitol, lactose, maltose, maltitol, dicalcium phosphate, talc, silicon dioxide, nonionic surfactant, bromphenol blue, and bromocresol green.

In some embodiments the one or more water-soluble polymers are independently selected from the list consisting of hydroxypropyl methylcellulose (HPMC), methylcellulose (MC), carboxymethylcellulose (CMC), such as sodium carboxymethyl cellulose (CMC), an alginate, such as sodium alginate, carrageenan, pectin, hydroxypropyl cellulose (HPC), hydroxyethyl cellulose (HEC), hydroxyethyl methylcellulose (HEMC), chitosan, trimethyl chitosan, hyaluronic acid, polycarbophil, carbomer, polyethylene oxide, and methacrylic acid derivative; derivatives and salts thereof.

In some embodiments the one or more water-soluble polymers is HPMC, such as a highly hydrophilic, lower molecular weight HPMC, such as a HPMC with a viscosity less than 3000 mPa-s (or cP), such as with a viscosity less than 1000 mPa-s (or cP), such as less than 750 mPa-s (or cP), such as less than 500 mPa-s (or cP), such as less than 400, 350, 300, 250, 200, 150, 120, 100, or 80 mPa-s (or cP). In some embodiments the small-molecule weak acid (WA), weak acid surfactant (WAS) or salt thereof is selected from a carbonic acid, monovalent metal phosphate salts, citric acid, succinic acid, oleic acid, caprylic acid, capric acid, decanoic acid, lauric acid, phosphotidylcholine, salicylic acid, methylsalicylic acid, ethylene diamine tetraacetic acid, acetic acid, cholic acid, deoxycholic acid, glycolic acid, glycocholic acid, glycodeoxycholic acid, taurocholic acid, taurodihydrofusidic acid, sodium caprate, sodium decanoate, sodium caprylate, sodium octanoate, sodium laurate, sodium dodecyl sulfate, dioctyl sodium sulfosuccinate, glyceryl behenate, glyceryl dibehenate, glyceryl monostearate, sodium N-[8-(2-hydroxybenzoyl)aminocaprylate], salcaprozate sodium, SNAC, N-(5-chlorosalicyloyl)-8-aminocaprylic acid, 5-CNAC, N-[10-(2-hydroxybenzoyl)aminocaprate], N-[10-(2-hydroxybenzoyl)aminodecanoate], sodium lauryl sulfate, sodium stearyl fumarate, sodium deoxycholate.

In some embodiments, the pharmaceutical solid dosage form further comprises one or more additional ingredients that protect the biomacromolecule from enzymatic digestion, such as sacrificial enzyme substrates. Sacrificial enzyme substrates can be selected from protease inhibitors or small peptides. The protease inhibitor can be selected from aprotinin, cysteine, threonine, asparagine, serpin, soybean trypsin inhibitor, or derivatives thereof. The small peptide can be selected from a dipeptide, tripeptide, tetrapeptide, pentapeptide, hexapeptide, heptapeptide, or octapeptide.

In some embodiments the solid dosage form is substantially homogenous without a coating or barrier.

In some embodiments the solid dosage form is coated with one or more polymers in order to target specific areas of the gastrointestinal tract, such as the stomach, duodenum, jejunum, ileum, cecum, or colon.

In some embodiments the solid dosage form is coated with one or more pH-dependent polymers.

In some embodiments the solid dosage form is coated with one or more pH-independent polymers.

In some embodiments the biomacromolecule following exposure of the pharmaceutical solid dosage form to a solution of HCl pH 1.2 with or without pepsin is protected from degradation to an extent such that the area under curve (AUC) from 0-60 min is more than 830%-min for intact biomacromolecule content vs. time, such as more than 1000%-min, such as more than 1500%-min.

### Applications

The dosage forms disclosed in this specification may be used in a variety of applications. In some embodiments, the dosage form is used in a human therapeutic. In some embodiments, the dosage form is used in an animal therapeutic. In some embodiments, the dosage form is used in a pet therapeutic. In some embodiments, the dosage form is used in a farm animal therapeutic, such as a nutritional application for swine, poultry or cattle. In some embodiments, the dosage form is used in a human nutrition application. In some embodiments, the dosage form is used in a human dietary supplement. In some embodiments, the dosage form is used in an animal nutrition application. In some embodiments, the dosage form is used in an animal dietary supplement. In some embodiments, the dosage form is used in a pet nutrition application. In some embodiments, the dosage form is used in a farm animal nutrition application.

In some embodiments, a dosage form disclosed in this specification is administered to a subject (human or animal) to treat a condition that can be treated by a therapeutically active biomacromolecule. In some embodiments, a dosage form disclosed in this specification is administered to a subject (human or animal) to treat a condition that can be treated by one or more enzymes disclosed in this specification. In some embodiments, the condition comprises a gastrointestinal disease. In some embodiments, the condition comprises an oral condition. In some embodiments, the condition comprises a digestive or gut condition. In some embodiments, the condition comprises a metabolic disorder. In some embodiments, the condition comprises an inherited metabolic disorder. In some embodiments, the condition comprises phenylkenoturia (PKU) or tyrosinemia. In some embodiments, the condition comprises exocrine pancreatic insufficiency. In some embodiments, the condition (e.g., an exoxrine pancreatic insufficiency) results from cystic fibrosis, pancreatitis, pancreatic cancer, diabetes, inflammatory bowel disease (IBD), irritable bowel syndrome (IBS), celiac disease, or a condition related to maldigestion and/or malabsorption. In some embodiments, the condition comprises homocystinuria. In some embodiments, the condition comprises maple syrup urine disease. In some embodiments, the condition is associated with gluten management, such as gluten intolerance, a gluten allergy or celiac disease. In some embodiments, the condition comprises lysosomal storage disorder.

In some embodiments, the dosage form is administered to a subject (i.e., a human or animal) to treat a condition disclosed in the preceding paragraph and the therapeutically active biomacromolecule comprises an enzyme (e.g., an enzyme disclosed in this specification).

### EXAMPLES

### Example 1

The following powder ingredients were weighed:
Bovine lactoferrin 15 g (Parchem)
Emprove sodium caprylate 150 g (Merck Millipore Sigma)
Avicel PH 101 microcrystalline cellulose 100.5 g (IFF)
METHOCEL K100LV HPMC (22.9% methyl, 10.1% hydroxypropyl, 104 mPa-s viscosity, 71.4% through 230 mesh screen) 30 g (IFF)
Bromocresol green 3 g (Merck Millipore Sigma)
Alubra sodium stearyl fumarate 1.5 g (IFF)
Total powder blend for tableting 300 g

The formulation ingredients, except for Alubra sodium stearyl fumarate, were blended together by placing the ingredients in a glass jar, capping, and blending via a Turbula mixer for 10 min. Alubra sodium stearyl fumarate was added after the first 10 min of blending and then blended for 1 min. Biconvex 2-mm round minitablet tooling was used to compact the powder blend using 14 of 16 stations on a Manesty Beta rotary press. Turret speed was set at 13 rpm in order to observe die fill during tableting, and compression force was targeted at 500 pounds (lb.) or 2.2 kN. The minitablet formulation was then sealed and stored in a Ziploc bag at ambient room conditions for a duration of at least overnight prior to analysis.

Twenty minitablets were measured individually to determine single-unit weight, diameter, and thickness. Single-unit weight was determined via analytical balance. Single-unit diameter and thickness were determined using a caliper. The individual minitablet dimension measurements as well as the tooling cup dimension specifications provided by the minitablet tooling manufacturer, Natoli Engineering, were used to determine single-unit surface area (SA), single-unit surface area-to-volume (SA/Vol), single-unit volume, and single-unit density. Further details are described in the sections below on the methodology used to determine single-unit SA, single-unit SA/Vol, single-unit volume, single-unit density, intact lactoferrin content vs. time, and area under the intact lactoferrin content vs. time curve (AUC_{0-60 min}). Single-unit SA/Vol and single-unit density for Example 1 composition were 3.49±0.098 mm⁻¹ and 1.32±0.053 g/cm³, respectively. AUC_{0-60 min} was 1840.00±513.400%-min.

### Determination of Single-Unit Physical Features

Since the minitablets were biconvex rather than flat-faced, minitablet surface area (SA in mm²) was calculated according to the equation:

$$Single Unit S A ( {mm}^{2} ) = ( 2 \times Tooling Cup Area ) + 2 π r ( r + t ) - 2 π {r}^{2}$$

where t is minitablet band thickness, and r is minitablet radius. The final 2πr² term in the equation accounts for and removes the combined surface area of the two embedded flat faces, since the minitablets are biconvex rather than flat-faced.

Minitablet radius was calculated by dividing minitablet diameter in half.

Tooling cup area as specified by the minitablet tooling manufacturer, Natoli Engineering, is 3.3800 mm².

Minitablet band thickness was calculated according to the equation:

$$Single Unit Band Thickness ( mm ) = Total Single Unit Thickness - 2 ( Cup Depth )$$

Minitablet tooling cup depth, as specified by Natoli Engineering, is 0.2800 mm.

Minitablet surface area-to-volume ratio (SA/Vol in mm⁻¹) was calculated according to the equation:

$$Single Unit \frac{S A}{Vol} ( {mm}^{- 1} ) = \frac{( 2 \times Tooling Cup Area ) + 2 π r ( r + t ) - 2 π {r}^{2}}{( 2 \times Tooling Cup Volume ) + π {r}^{2} t}$$

Minitablet tooling cup volume, as specified by Natoli Engineering, is 0.4100 mm³.

Minitablet volume (cm³) was calculated according to the equation:

$$Single Unit Vol ( {cm}^{3} ) = \frac{Single Unit S A}{Single Unit \frac{S A}{Vol}} \times {( \frac{1}{10} )}^{3}$$

Minitablet density (g/cm³) was calculated according to the equation:

$$Single Unit Density ( \frac{g}{{cm}^{3}} ) = \frac{( \frac{Single Unit Weight ( mg )}{1000} )}{Single Unit Vol ( {cm}^{3} )}$$

### Determination of Intact Lactoferrin Content

200 mg of minitablets (typically containing 5% lactoferrin) were added to 10 mL pH 1.2 HCl+pepsin in a 50 mL centrifuge tube. 200 mg of minitablets is approximately 50 minitablets. pH 1.2 HCl+pepsin was prepared according to the United States Pharmacopeia recipe for simulated gastric fluid (SGF). The tube was capped and placed on tilted roller (rolling at 6-7 rpm and equilibrated to 37° C.) for the designated duration (0, 10, 20, 30, 40, 50, or 60 min). The entire roller set up is situated in a constant-temperature chamber, so the temperature can be controlled to 37° C. The roller is tilted 7°, so the 10 ml of pH 1.2 HCL+pepsin pools towards the bottom of the tube as it is rolled, submerging and gently agitating the dosage being tested. The centrifuge tube was removed from the roller at the specified time point, and 20 ml of pH 9.0 Neutralizing Buffer were added to the centrifuge tube to neutralize to pH 7, followed by vortexing for 1 min. pH 9.0 Neutralizing Buffer was prepared by dissolving 14.2 g sodium phosphate dibasic anhydrous in 1 L deionized water. Two sets of roller time point samples were tested per day, n=3 repetitions per roller time point sample set. i.e. 6 total samples could be tested each day. Each neutralized sample was then frozen. Each frozen neutralized sample was removed from the freezer and rolled overnight on a roller situated in a refrigerator (4.8° C.) to thaw. Chromogen, Working Buffer Stock, Washing Buffer Stock, and Stop solutions (all supplied with the ELISA assay kit) were removed from the storage refrigerator (6° C.) the morning prior to analysis and equilibrated to ambient room temperature. Working Buffer Stock was diluted 5× prior to use, 16 ml milli-Q water+4 mL Working Buffer Stock. Washing Buffer Stock was diluted 20× prior to use, 19 mL milli-Q water+1 mL Washing Buffer Stock. Four 2-mL centrifuge tubes, Tubes A through D, were prepared for each sample. More or less tubes were used depending upon the concentration of lactoferrin used. 0.9 mL of diluted Working Buffer was pipetted into each tube. 0.1 mL of thawed neutralized roller sample was pipetted into Tube A, followed by drawing in and expectorating from the pipette 5 times and then vortexing for couple of seconds. 0.1 mL from Tube A was pipetted into Tube B, followed by drawing in and expectorating from the pipette 5 times and then vortexing for a couple of seconds. 0.1 mL from Tube B was pipetted into Tube C, followed by drawing in and expectorating from the pipette 5 times and then vortexing for a couple of seconds. 0.1 mL from Tube C was pipetted into Tube D, followed by drawing in and expectorating from the pipette 5 times and then vortexing for a couple of seconds. 0.1 mL from Tube D was pipetted into a well of the ELISA assay plate. (Bovine Lactoferrin ELISA Kit, ab274406, by Abcam. See https://www.abcam.com/bovine-lactoferrin-elisa-kit-ab274406.html for more information about the Bovine Lactoferrin ELISA assay plate.) The above steps were repeated for each of the remaining five thawed neutralized roller samples. The assay plate was covered with microplate sealing tape (Thermofisher Scientific, part number 9503130), and the plate was placed into the Tecan plate reader. The plate was shaken for 3 minutes and held for 27 minutes at 23° C. The assay plate was removed from the Tecan instrument, and the wells were washed out 5 times by filling the wells with diluted Washing Buffer and then tapping out onto paper towels to empty. For the last rinse, the diluted Washing Buffer was left in the wells for 2 min before tapping out onto the paper towel to empty. The tube containing antibody (supplied with the ELISA kit) was taken out of the storage refrigerator (6° C.) and equilibrated to ambient room temperature (approximately 0.5 h). 0.990 mL of diluted Working Buffer was added into a new small vial. 0.010 mL equilibrated antibody solution was added to the vial. 0.100 ml of the prepared solution was added to each of the 5× rinsed wells of the assay plate. (Note: One tube of diluted antibody solution is needed per well strip.) The assay plate was again covered with the microplate tape and placed into the Tecan for 3 minutes of shaking and holding for 27 minutes at 23° C. The assay plate was removed from the Tecan, and the wells were washed out 5 times with diluted Washing Buffer. For the last rinsing cycle, diluted Washing Buffer was left in the wells for 2 minutes before emptying. 0.100 ml of chromogen solution (equilibrated to ambient room temperature) was added to each of the 5× rinsed wells of the assay plate. The assay plate was covered with the sealing tape and placed into the Tecan. The assay plate was shaken for 3 minutes and then held for 7 minutes at 23° C. The assay plate was removed from the Tecan, and 0.100 ml of Stop Solution (supplied with the ELISA assay kit) was added to the well. The assay plate was immediately placed back into the Tecan, this time without microplate tape. The plate reader was set to hold for 1 minute, and the UV spectrophotometer then analyzed the wells at 450 nm wavelength. Intact lactoferrin content data was supplied upon completion of analysis. The intact lactoferrin content vs. time curve from 0-60 min was plotted, and the area under the intact lactoferrin content vs. time curve, AUC_{0-60 min} was calculated.

### Preparation of Calibration Standards for Determination of Intact Lactoferrin

Approximately 17 mg lactoferrin was added to 100 ml milliQ water in a vial that was then capped and rolled under refrigeration (4.8° C.) for at least 2 h. This served as the Lactoferrin Stock solution used to make Calibration Standards. 0.1 mL of Lactoferrin Stock was diluted with 0.9 mL Diluted Working Buffer to make a 10× Dilution. The 10× Dilution was mixed together by pulling and expectorating from the pipette 5 times, followed by vortexing for a couple of seconds. 0.1 mL from 10× Dilution was further diluted with 0.9 mL Diluted Working Buffer to make a 100× Dilution. The 100× Dilution was mixed together by drawing in and expectorating from the pipette 5 times, followed by vortexing for a couple of seconds. Eight 2-mL centrifuge tubes were prepared for the Calibration Standards, labeled 1-8. 1 mL of Diluted Working Buffer was pipetted into Tubes 1 and 8. 0.400 mL of Diluted Working Buffer was pipetted into Tubes 2-7. 0.050 mL of 100× Dilution was pipetted into Tube 1 and mixed. 0.040 mL of 100× Dilution was pipetted into Tube 8 and mixed. 0.400 mL from Tube 1 was pipetted into Tube 2, rinsing the pipette tip 5 times by drawing in and expectorating, followed by vortexing for a couple of seconds. 0.400 mL from Tube 2 was pipetted into Tube 3, rinsing the pipette tip 5 times by drawing in and expectorating, followed by vortexing for a couple of seconds. Serial dilutions were carried out through Tube 6. Tube 7 just contained Diluted Working Buffer blank solution (no lactoferrin). 0.100 ml of each calibration standard was pipetted into each well in that particular row of the ELISA assay plate and each of these wells was prepared and analyzed accordingly as described earlier. This row was used to generate the calibration curve.

### Comparative Example A

The following powder ingredients were weighed:
Bovine lactoferrin 15 g
Emprove sodium caprylate 150 g
Avicel PH 102 microcrystalline cellulose 132 g (IFF)
Cabosil M5P silicon dioxide 1.5 g (Cabot)
Alubra sodium stearyl fumarate 1.5 g (IFF)
Total powder blend for tableting 300 g

The formulation ingredients, except for Alubra sodium stearyl fumarate, were blended together 10 min, and then Alubra sodium stearyl fumarate was added and blended 1 min, as in Example 1. Biconvex 7.94-mm round tablet tooling was used to compact the powder blend using 8 of 16 stations on a Manesty Beta rotary press. Turret speed was set at 13 rpm in order to observe die fill during tableting, and compression force was targeted at 5000 pounds (lb.) or 22.2 kN. As was done for Example 1, the tablet formulation was then sealed in a Ziploc bag and stored at ambient room conditions at least overnight prior to analysis.

The weight and dimensions of 20 individual tablets were measured similarly to Example 1. For Comparative Example A, however, individual tablet dimension measurements were based upon biconvex 7.94-mm round tooling cup dimension specifications provided by Natoli Engineering. 7.94-mm tooling cup area, cup volume, and cup depth were 51.8257 mm², 21.6309 mm³, and 0.8636 mm, respectively. Single-unit surface area (SA), single-unit surface area-to-volume (SA/Vol), single-unit volume, and single-unit density were determined using the equations already provided for Example 1. Intact lactoferrin was determined in pH 1.2 HCl with pepsin via ELISA measurement of intact lactoferrin content, using the methodology described for Example 1. Single-unit SA/Vol and single-unit density for Comparative Example A composition were 1.00±0.003 mm⁻¹ and 1.18±0.008 g/cm³, respectively. AUC_{0-60 min} was 1801.67±352.628%-min.

### Comparative Example B

Multiparticulates comprised sucrose nonpareil beads coated in a Glatt GPCG-1 fluidized bed coater via bottom spray (started at 3 g/min spray rate and gradually increased up to 5.7 g/min) with a Wurster column insert using a 10% aqueous solution containing METHOCEL™ E5 LV and lactoferrin in a 1:1 weight ratio. The liquid forming the solution was deionized (DI) water. After 2.5 h of processing at 45° C. outlet temperature, 400 g of nonpareil sucrose beads were coated to a final weight of 436 g.

Since the multiparticulates were spherical, SA was measured according to the equation:

$$S A ( {mm}^{2} ) = 4 π {r}^{2}$$

and Vol was measured according to the equation:

$$Vol ( {mm}^{3} ) = \frac{4}{3} π {r}^{3}$$

SA/Vol was measured according to the equation:

$$\frac{S A}{Vol} ( {mm}^{- 1} ) = \frac{( 4 π {r}^{2} )}{( \frac{4}{3} π {r}^{3} )}$$

It was not feasible to load the EMPROVE sodium caprylate onto the multiparticulates at the needed level, so the sodium caprylate powder was added along with the coated multiparticulates into pH 1.2 HCl with pepsin to determine intact lactoferrin vs. time. Intact lactoferrin was quantitated using the ELISA methodology described for Example 1. Single-unit SA/Vol and single-unit density for Comparative Example B composition were 6.47±0.258 mm⁻¹ and 1.64±0.321 g/cm³, respectively. AUC_{0-60 min} was 263.33±241.109%-min.

### Comparative Example C

The following powder ingredients were weighed:
Bovine lactoferrin 15 g
Emprove sodium caprylate 150 g
Avicel PH 101 microcrystalline cellulose 130.5 g
Bromocresol green 3 g
Alubra sodium stearyl fumarate 1.5 g
Total powder blend for tableting 300 g

The formulation ingredients, except for Alubra sodium stearyl fumarate, were blended together 10 min, and then Alubra sodium stearyl fumarate was added and blended 1 min, as in Example 1. Biconvex 2-mm round minitablet tooling was used to compact the powder blend using 14 of 16 stations on a Manesty Beta rotary press, as in Example 1. Turret speed was set at 13 rpm in order to observe die fill during tableting, and compression force was targeted at 500 pounds (lb.) or 2.2 kN, also as was done for Example 1. And as was done for Example 1, the minitablet formulation was then sealed in a Ziploc bag and stored at ambient room conditions at least overnight prior to analysis.

The weight and dimensions of 20 individual minitablets were measured similarly to Example 1. Single-unit surface area (SA), single-unit surface area-to-volume (SA/Vol), single-unit volume, and single-unit density were determined using the equations already provided for Example 1. Intact lactoferrin was determined in pH 1.2 HCl with pepsin via ELISA measurement of intact lactoferrin content, using the methodology described for Example 1. Single-unit SA/Vol and single-unit density for Comparative Example C composition were 3.61±0.149 mm⁻¹ and 1.50±0.097 g/cm³, respectively. AUC_{0-60 min} was 977.50±312.630%-min.

TABLE 1

| | | | | Intact | Intact |
|---|---|---|---|---|---|
| | | | Unit | Content | Content at |
| Delivery | | SA/Vol | Density | AUC_{0-60 min} | 60 min |
| System Type | Role | (mm⁻¹) | (g/cm³) | (%-min) | (%) |
| Standard | Compar- | 1.00 ± | 1.18 ± | 1801.67 ± | 3.33 ± |
| Tablet | ative | 0.003 | 0.008 | 352.628 | 7.202 |
| | Example A | | | (n = 6) | (n = 6) |
| Multipar- | Compar- | 6.47 ± | 1.64 ± | 263.33 ± | 0.00 ± |
| ticulate | ative | 0.258 | 0.321 | 241.109 | 0.000 |
| | Example B | | | (n = 3) | (n = 3) |
| Multi-Site | Compar- | 3.61 ± | 1.50 ± | 977.50 ± | 4.17 ± |
| | ative | 0.149 | 0.097 | 312.630 | 2.401 |
| | Example C | | | (n = 6) | (n = 6) |
| Multi-Site | Example 1 | 3.49 ± | 1.32 ± | 1840.00 ± | 24.33 ± |
| Microenvi- | | 0.098 | 0.053 | 513.400 | 13.337 |
| ronment | | | | (n = 6) | (n = 6) |

AUC_{0-60 min} in Table 1 is area under the intact lactoferrin vs. time profile from 0 to 60 min.

### Example 2

The following powder ingredients were weighed for Example 2 (a):
Lipase mix 2.5 g (IFF)
Emprove sodium caprylate 25 g (Merck Millipore Sigma)
Avicel PH 101 microcrystalline cellulose 17 g (IFF)
METHOCEL K100LV HPMC (22.9% methyl, 10.1% hydroxypropyl, 104 mPa-s viscosity, 71.4% through 230 mesh screen) 5 g (IFF)
Cabosil M5P silicon dioxide 0.25 g (Cabot)
Alubra sodium stearyl fumarate 0.25 g (IFF)
Total powder blend for tableting 50 g

The following powder ingredients were weighed for Example 2 (b):
Lipase mix 2.5 g (IFF)
Emprove sodium caprylate 12.5 g (Merck Millipore Sigma)
Avicel PH 101 microcrystalline cellulose 29.5 g (IFF)
METHOCEL K100LV HPMC (22.9% methyl, 10.1% hydroxypropyl, 104 mPa-s viscosity, 71.4% through 230 mesh screen) 5 g (IFF)
Cabosil M5P silicon dioxide 0.25 g (Cabot)
Alubra sodium stearyl fumarate 0.25 g (IFF)
Total powder blend for tableting 50 g

The formulation ingredients, except Alubra sodium stearyl fumarate, were placed on a #20 mesh screen and hand-sieved through the screen. The sieved ingredients were blended together by placing the ingredients in a glass jar, capping, and blending via a Turbula mixer for 10 min. Alubra sodium stearyl fumarate was added after the first 10 min of blending and then blended for 1 min. Biconvex 2-mm round minitablet tooling, same as that described in Example 1, was used to compact the powder blend utilizing 2-3 stations at a time on a Manesty Beta rotary press. Due to cost and small quantity of raw material, blended powder was hand-fed into the 2-3 sets of dies available on the turret while the tableting machine was turned off, and the press was subsequently jogged at approximately 2.2 kN of force, with turret speed set at 13 rpm, to compress the powders to minitablets. The minitablets were then collected from the turret using a spatula, and sealed and stored in a Ziploc bag at least overnight at ambient lab conditions before analysis.

Twenty minitablets containing lipase mix were measured individually to determine single-unit weight, diameter, and thickness using the methodology described in Example 1. SA/Vol and Unit Density were calculated using the equations described in Example 1 and are shown in Table 2.

TABLE 2

| | | | Unit |
|---|---|---|---|
| Delivery | | SA/Vol | Density |
| System Type | Role | (mm⁻¹) | (g/cm³) |
| Multi-Site | Example 2(a) | 2.88 ± 0.069 | 1.28 ± 0.050 |
| Microenvironment | | | |
| Multi-Site | Example 2(b) | 2.87 ± 0.066 | 1.32 ± 0.071 |
| Microenvironment | | | |

### Example 3

The following powder ingredients were weighed:
Semaglutide 50 mg (BOC Sciences)
Emprove sodium caprylate 500 mg (Merck Millipore Sigma)
Avicel PH 101 microcrystalline cellulose 345 mg (IFF)
METHOCEL K100LV HPMC (22.9% methyl, 10.1% hydroxypropyl, 104 mPa-s viscosity, 71.4% through 230 mesh screen) 100 mg (IFF)
Alubra sodium stearyl fumarate 5 mg (IFF)
Total powder blend for tableting 1000 mg

The formulation ingredients, except Alubra sodium stearyl fumarate, were blended together by placing the ingredients in a glass jar, capping, and blending via a Turbula mixer for 10 min. Alubra sodium stearyl fumarate was added after the first 10 min of blending and then blended for 1 min. Biconvex 2-mm round minitablet tooling, same as that described in Example 1, was used to compact the powder blend utilizing 2-3 stations at a time on a Manesty Beta rotary press. Due to cost and small quantity of raw material, blended powder was hand-fed into the 2-3 sets of dies available on the turret while the tableting machine was turned off, and the press was subsequently jogged at approximately 2.2 kN of force, with turret speed set at 13 rpm, to compress the powders to minitablets. The minitablets were then collected from the turret using a spatula, and sealed and stored in a Ziploc bag in the −20° C. freezer for a duration of at least overnight prior to analysis. In order to experimentally adjust the amount of sodium caprylate, a placebo version of the minitablets was produced, running the press in full operation mode, using the parameters described in Example 1. This allowed combinations of active and placebo minitablets to vary the sodium caprylate dosage while holding the semaglutide dosage constant.

Twenty minitablets containing semaglutide were measured individually to determine single-unit weight, diameter, and thickness using the methodology described in Example 1.

### Determination of Intact Semaglutide Content

60 mg of minitablets (containing 5% semaglutide) were added to 10 mL pH 1.2 HCl+pepsin in a 50 mL centrifuge tube. pH 1.2 HCl+pepsin was prepared according to the United States Pharmacopeia recipe for simulated gastric fluid (SGF). 60 mg of minitablets contains approximately 3 mg semaglutide and is approximately 12 minitablets. The placebo version of the minitablets (containing all ingredients except semaglutide) was measured and added, if needed to adjust the level of sodium caprylate. For example, 140 mg of placebo minitablets were added along with 60 mg semaglutide minitablets in order increase the sodium caprylate dosage to 100 mg while keeping the semaglutide dosage constant at 3 mg. Example 3 (a) comprises 60 mg semaglutide minitablets plus 140 mg placebo minitablets. Example 3 (b) comprises 60 mg semaglutide minitablets only.

The 50 mL centrifuge tube was capped and placed on a tilted roller (rolling at 6-7 rpm) for the designated duration (0, 10, 20, 30, 40, or 50 min). As in Example 1, the entire roller set up is situated in a constant-temperature chamber, so temperature can be controlled to 37° C. The roller is tilted 7°, so the 10 mL of pH 1.2 HCL+pepsin pools towards the bottom of the tube as it is rolled, submerging and gently agitating the formulation being tested. The centrifuge tube was removed from the roller at the specified time point, and 20 ml of pH 9.0 Neutralizing Buffer were added to the centrifuge tube to neutralize to pH 7, followed by vortexing for 1 min. The neutralized samples were then placed on a roller situated in a refrigerator and rolled overnight for ELISA analysis starting the following morning. The temperature in that refrigerator was set to 4° C. The intact semaglutide content vs. time curve from 0-50 min was plotted, and the area under the intact semaglutide content vs. time curve, AUC_{0-50 min} was calculated.

### ELISA Analysis for Determination of Intact Semaglutide

The ELISA kit from OriGene Technologies (Cat #: S-1530) was used to determine the amount of intact semaglutide. The ELISA kit contained an immunoplate with 12 eight-well strips, antiserum powder, biotinylated tracer, ELISA buffer, streptavidin-HRP (horseradish peroxidase), TMB (3,3′,5,5′-tetramethylbenzidine) substrate solution, TMB substrate buffer, and 2N HCl as the stop solution. The protocol included with the kit was followed during testing. In each run of the test, one strip of immunoplate was used for applying calibration standards and another strip was used for applying samples. In the strip for the standards, the first well had 75 μL of ELISA buffer and the remaining wells had 50 μL of semaglutide standards of varying concentrations and 25 μL of antiserum. In the strip for the samples, the wells had 50 μL of control or test samples and 25 μl of antiserum, respectively. The strips were then incubated in a Tecan microplate reader (INFINITE 200 PRO) at 23° C. for one hour. Afterwards, 25 μL of biotinylated tracer solution was added to all wells. The plate was transferred to the microplate reader to incubate for two hours. The solutions in the wells were decanted after incubation and the strips were washed with ELISA buffer five times. After washing, 100 μL of streptavidin-HRP solution was added to all wells, and the strips were incubated again in the microplate reader for one hour. The strips were then washed again five times using the ELISA buffer. TMB chromogenic solution was then added to all the wells by pipetting 100 μL into each well. After 10 minutes of incubation in the microplate reader, 100 μL of stop solution were added to all the wells. UV absorbance of all the wells was read at 450 nm after 5 minutes of incubation.

The absorbance of standards after subtraction from that of blank was plotted against their concentrations on a semi-log scale, in which the log of UV absorbance was on y-axis and standard concentrations were on x-axis. The data were fitted by four-parameter logistic regression and the derived parameters were used to calculate the intact peptide concentrations from the UV readings of controls and samples after blank subtraction.

The impact of pH 1.2 HCl+pepsin on semaglutide was first studied by adding 100 μl of semaglutide solution (1 mg/ml in ELISA buffer) to 900 μL of pH 1.2 HCl+pepsin and the solution was then placed on a roller. The same semaglutide solution was also added to 900 μL of deionized water as a control. After mixing for a given time (1, 5, 15, 30, 60 min), both the pH 1.2 HCl+pepsin solution and the control solution were diluted 10,000× with ELISA buffer. The dilution was done in 4 serial steps of 10× ratio by adding 100 μL of the peptide solutions to 900 μL of ELISA buffer. The solutions were then added to the strip of immunoplate for concentration measurement of intact peptide. The percentage of the intact peptide after treatment with pH 1.2 HCl+pepsin was determined as:

$$\% intact semaglutide = concentration in pH 1.2 HCl + pepsin solution / concentration in control solution \times 100$$

After pH 1.2 HCl+pepsin treatment and neutralization, aliquots of the tested formulations were filtered by PVDF syringe filters into minicentrifuge tubes. The filtered solutions were diluted with ELISA buffer in 4 serial steps. The dilution ratio in each step is 10× by adding 100 μL of the sample solution into 900 μL of ELISA buffer. The sample solutions were applied to the strip of immunoplate for determination of concentrations of the intact peptide. A semaglutide control (Fisher cat #: 50-225-9952) was also applied to the same strip. The concentration of intact semaglutide in the solution was determined as:

Intact semaglutide concentration=measured concentration of sample/measured concentration of the control×concentration of the control from preparation

$$\% intact semaglutide = concentration of intact semaglutide from ELISA / concentration of semaglutide from preparation \times 100$$

### Preparation of Calibration Standards for Determination of Intact Semaglutide

Semaglutide (5 mg) received from BOC Sciences was dissolved in 5 mL of ELISA buffer to prepare 1 mg/mL stock standard solution. The 5-mL solution was separated into multiple vials in 120-μL aliquots. All the aliquots were stored in a freezer at −18° C. One aliquot of the stock standard solution was transferred to the refrigerator where the samples were stored before the of day of conducting ELISA assay. The aliquot was diluted by 1,000× with ELISA buffer in three serial steps where 100 μL of the standard solution was added into 900 μL of the buffer. The 1 μg/mL standard solution was then diluted in six serial steps to make the standards to be applied to the strip of immunoplate. The first step was to dilute the standard 10× by adding 100 μL of the standard solution to 900 μL ELISA buffer to make the most concentrated standard (S1). The second step was to add 200 μl of S1 to 600 μL of ELISA buffer to make S2. The third, fourth, fifth, and sixth steps were to make the standards in decreasing concentrations by adding 200 μL of S2 to 600 μL of ELISA buffer to make S3; adding 200 μL of S3 to 600 μL of ELISA buffer to make S4; adding 200 μL of S4 to 600 μL of ELISA buffer to make S5; adding 200 μl of S5 to 600 μL of ELISA buffer to make S6. S7 was the buffer without the peptide serving as the zero-concentration standard. In one strip of immunoplate, the first well had 75 μL of ELISA buffer as the blank. The second to the eighth well had 50 μL of S1 to S7 standards and 25 μL of antiserum, respectively.

### Preparation of Factoring Standard of Intact Semaglutide

Semaglutide from Fisher Scientific (cat #: 50-225-9952) was used as factoring standard. The peptide was received in 5 mg quantity and dissolved in 5 mL of ELISA buffer. The solution was distributed into multiple vials. Each vial had 120 μL aliquot of the solution. All the aliquots were stored in a freezer at −18° C. One aliquot was transferred from the freezer to the refrigerator before the day of testing. The same refrigerator also had the samples for testing. The aliquot was diluted 100,000× by adding 100 μL of the control solutions to 900 μL of ELISA buffer in five serial steps. The diluted factoring standard control solution was applied to the same strip of immunoplate where the sample solutions would be applied.

### Disintegration Testing

Disintegration testing was conducted using the Sotax DT50 Disintegration Tester (Sotax Corp., Westborough, MA USA)). Using 1000 ml beakers from Sotax (3061-1) with the induction plate (14728-01) placed at the bottom of the beaker, 800 ml of degassed pH 1.2 HCl was added and warmed to 37° C. The basket type used was SKx with a trigger value set to 0.5 mm. The basket was assembled with a 20-mesh screen, glass tube, each glass tube containing 200 mg of minitablets, and the sensor disc (red side facing down) placed on top of the minitablets. Once the basket was attached to the holder on the main unit, testing was run under “Direct Test” on the main menu. Default testing parameters were used. End point was automatically recorded once the sensor disc reached the 0.5-mm trigger value.

TABLE 3

| Delivery | | Sema- | | | Unit | Intact Content | Intact | |
|---|---|---|---|---|---|---|---|---|
| System | | glutide | WAS | SA/Vol | Density | AUC_{0-50 min} | Content at | Disintegration |
| Type | Role | (mg) | (mg) | (mm⁻¹) | (g/cm³) | (%-min) | 30 min (%) | Time (min) |
| Multi-Site | Ex | 3 | 100 | 3.31 ± | 1.25 ± | 4964.52 ± 437.435 | 88.08 ± 6.170 | 14.03 ± 1.797 |
| Microenvi- | 3(a) | | | 0.128 | 0.124 | (n = 3) | (n = 3) | (n = 12) |
| ronment | | | | | | | | |
| Multi-Site | Ex | 3 | 30 | 3.31 ± | 1.25 ± | 5012.11 | 86.81 | 14.03 ± 1.797 |
| Microenvi- | 3(b) | | | 0.128 | 0.124 | (n = 1) | (n = 1) | (n = 12) |
| ronment | | | | | | | | |

In Table 3, the AUC_{0-50 min} is area under the intact semaglutide vs. time profile from 0 to 50 min. Also, to conserve semaglutide, the disintegration time testing was conducted on the placebo version of the multi-site microenvironment rather than the formulation containing the peptide API.

1. A pharmaceutical solid dosage form for the treatment of a condition by gastrointestinal delivery comprising multiple single units, each single unit comprising:
a) one or more therapeutically active biomacromolecules;
b) one or more water-soluble polymers in an amount of not more than about 50 weight %;
c) a small-molecule weak acid (WA), weak acid surfactant (WAS), or salt thereof in an amount of not more than about 75 weight %;
each single unit having a surface area to volume (SA/Vol) ratio greater than 1.0 mm⁻¹ and a single-unit density greater than 1.0 g/cm³; the solid dosage form comprising the therapeutically active biomacromolecule in a therapeutically effective amount derived from the combined multiple amount of each single unit.

2-3. (canceled)

4. The pharmaceutical solid dosage form according to claim 1, having a multiple-unit bulk density greater than 0.70 g/cm³.

5. The pharmaceutical solid dosage form according to claim 1, wherein the viscosity of the one or more water-soluble polymers is less than 3000 mPa-s (or cP).

6. The pharmaceutical solid dosage form according to claim 1, wherein each single unit has a surface area to volume (SA/Vol) ratio greater than 1.0 mm⁻¹.

7-9. (canceled)

10. The pharmaceutical solid dosage form according to claim 1, wherein the one or more biomacromolecules is independently selected from a protein, a peptide, a polypeptide, an oligopeptide, a synthetic polypeptide, a hormone, an insulin, a growth factor, a monoclonal antibody, a fusion protein, an enzyme, a therapeutic enzyme, a bispecific antibody, a multi-specific antibody, an antibody fragment, an interleukin, a cytokine, an antibody-drug conjugate, a glycoprotein, a viral protein, an oligonucleotide, a DNA fragment, an RNA fragment, messenger RNA, small interfering RNA, modified RNA, and any combination thereof.

11-12. (canceled)

13. The pharmaceutical solid dosage form according to claim 1, wherein the one or more biomacromolecules has a molecular weight higher than about 500 Da.

14. (canceled)

15. The pharmaceutical solid dosage form according to claim 1, wherein the one or more water-soluble polymers is independently selected from the list consisting of hydroxypropyl methylcellulose (HPMC), methylcellulose (MC), carboxymethylcellulose (CMC), an alginate, carrageenan, pectin, hydroxypropyl cellulose (HPC), hydroxyethyl cellulose (HEC), hydroxyethyl methylcellulose (HEMC), chitosan, trimethyl chitosan, hyaluronic acid, polycarbophil, carbomer, polyethylene oxide, methacrylic acid derivative, and derivatives and salts thereof.

16. The pharmaceutical solid dosage form according to claim 15, wherein the one or more water-soluble polymers is HPMC.

17. The pharmaceutical solid dosage form according to claim 1, wherein the small-molecule weak acid (WA), weak acid surfactant (WAS) or salt thereof is selected from a carbonic acid, monovalent metal phosphate salts, citric acid, succinic acid, oleic acid, caprylic acid, capric acid, decanoic acid, lauric acid, phosphotidylcholine, salicylic acid, methylsalicylic acid, ethylene diamine tetraacetic acid, acetic acid, cholic acid, deoxycholic acid, glycolic acid, glycocholic acid, glycodeoxycholic acid, taurocholic acid, taurodihydrofusidic acid, sodium caprate, sodium decanoate, sodium caprylate, sodium octanoate, sodium laurate, sodium dodecyl sulfate, dioctyl sodium sulfosuccinate, glyceryl behenate, glyceryl dibehenate, glyceryl monostearate, sodium N-[8-(2-hydroxybenzoyl)aminocaprylate], salcaprozate sodium, SNAC, N-(5-chlorosalicyloyl)-8-aminocaprylic acid, 5-CNAC, N-[10-(2-hydroxybenzoyl)aminocaprate], N-[10-(2-hydroxybenzoyl)aminodecanoate], sodium lauryl sulfate, sodium stearyl fumarate, and sodium deoxycholate.

18. The pharmaceutical solid dosage form according to claim 1, wherein the solid dosage form further comprises one or more additional ingredients that protect the biomacromolecule from enzymatic digestion.

19. The pharmaceutical solid dosage form according to claim 18, wherein the solid dosage form comprises a sacrificial enzyme substrate selected from a protease inhibitor and a small peptide.

20. The pharmaceutical solid dosage form according to claim 19, wherein the solid dosage form comprises a protease inhibitor selected from aprotinin, cysteine, threonine, asparagine, serpin, soybean trypsin inhibitor, and derivatives thereof.

21. The pharmaceutical solid dosage form according to claim 19, wherein the solid dosage form comprises a small peptide selected from a dipeptide, tripeptide, tetrapeptide, pentapeptide, hexapeptide, heptapeptide, and octapeptide.

22-25. (canceled)

26. The pharmaceutical solid dosage form according to claim 1, wherein the biomacromolecule following exposure of the pharmaceutical solid dosage form to a solution of HCl pH 1.2 with or without pepsin is protected from degradation to an extent such that the area under curve (AUC) from 0-60 min is more than 830%-min for intact biomacromolecule content vs. time.

27. The pharmaceutical solid dosage form according to claim 1, wherein the one or more biomacromolecules is combined with one or more small molecule APIs.

28. The pharmaceutical solid dosage form according to claim 10, wherein the one or more biomacromolecules comprise(s) an enzyme selected from a lipase, protease, amylase, enterokinase and carbohydrate enzyme.

29. (canceled)

30. A method for treating a subject in need of a biomacromolecule, wherein:
the method comprising (a) providing a solid oral dosage form as defined in claim 1, and (b) administering orally to a patient the solid oral dosage form; and
the biomacromolecule is selected from a protein, a peptide, a polypeptide, an oligopeptide, a synthetic polypeptide, a hormone, an insulin, a growth factor, a monoclonal antibody, a fusion protein, an enzyme, a therapeutic enzyme, a bispecific antibody, a multi-specific antibody, an antibody fragment, an interleukin, a cytokine, an antibody-drug conjugate, a glycoprotein, a viral protein, an oligonucleotide, a DNA fragment, an RNA fragment, messenger RNA, small interfering RNA, modified RNA, and any combination thereof.

31. The method according to claim 30, wherein the solid dosage form provides a pharmacokinetic profile of the active biomacromolecule with a T lag greater than 1.0 h and less than 16 h post-administration and a T max greater than (T lag+0.5 h) and less than 20 h post-administration.

32. The method according to claim 30, wherein the solid dosage form is administered to treat a condition in the subject selected from the group consisting of an oral condition; a digestive or gut condition; a metabolic disorder; phenylkenoturia; tyrosinemia; exocrine pancreatic insufficiency; homocystinuria; maple syrup urine disease; a condition associated with gluten management; and lysosomal storage disorder.

33. A process for the preparation of a pharmaceutical solid dosage form as defined in claim 1, which process comprises the steps of providing the components a), b) and c), and formulating the dosage form into a tablet or a capsule.
